(19) United States
(12) Patent Application Publication
(10) Pub. No.: US 2025/0002462 A1
(43) Pub. Date: Jan. 2, 2025

(54) SULFONE METHOD OF PREPARING PYRIMIDINE CYCLOHEXYL GLUCOCORTICOID RECEPTOR MODULATORS

(71) Applicant: Corcept Therapeutics Incorporated, Menlo Park, CA (US)

(72) Inventors: Hazel Joan Hunt, West Sussex (GB); Gary Patrick Reid, Menlo Park, CA (US); Jeffrey Mark Dener, Menlo Park, CA (US); Aaron Dumas, Hoddesdon (GB); Stephen Hyde, Hoddesdon (GB); Liam McLean, Hoddesdon (GB); Shaun Stairs, Hoddesdon (GB); Christopher Wise, Hoddesdon (GB)

(21) Appl. No.: 18/676,126

(22) Filed: May 28, 2024

Related U.S. Application Data
(60) Provisional application No. 63/504,824, filed on May 30, 2023.

(51) Int. Cl. C07D 239/54

(52) U.S. Cl. CPC C07D 239/54

(57) ABSTRACT

The present invention provides methods of preparing pyrimidine cyclohexyl glucocorticoid receptor modulators, methods of preparing intermediates of pyrimidine cyclohexyl glucocorticoid receptor modulators, and thioether compounds.

### CROSS-REFERENCES TO RELATED APPLICATIONS

This application claims the benefit of priority to U.S. Provisional Patent Application No. 63/504,824, filed 30 May 2023, the entirety of which is incorporated herein by reference.

### BACKGROUND

In most species, including man, the physiological glucocorticoid is cortisol (hydrocortisone). Glucocorticoids are secreted in response to ACTH (corticotropin), which shows both circadian rhythm variation and elevations in response to stress and food. Cortisol levels are responsive within minutes to many physical and psychological stresses, including trauma, surgery, exercise, anxiety and depression. Cortisol is a steroid and acts by binding to an intracellular, glucocorticoid receptor (GR). In man, glucocorticoid receptors are present in two forms: a ligand-binding GR-alpha of 777 amino acids; and, a GR-beta isoform which lacks the 50 carboxy terminal residues. Since these include the ligand binding domain, GR-beta is unable to bind ligand, is constitutively localized in the nucleus, and is transcriptionally inactive. The GR is also known as the GR-II.

The biologic effects of cortisol, including those caused by hypercortisolemia, can be modulated at the GR level using receptor modulators, such as agonists, partial agonists and antagonists. Several different classes of agents are able to inhibit the physiologic effects of GR-agonist binding. These antagonists include compositions which, by binding to GR, inhibit the ability of an agonist to effectively bind to and/or activate the GR. One such known GR antagonist, mifepristone, has been found to be an effective anti-glucocorticoid agent in humans (Bertagna (1984) J. Clin. Endocrinol. Metab. 59:25). Mifepristone binds to the GR with high affinity, with a dissociation constant (Kd) of 10-9 M (Cadepond (1997) Annu. Rev. Med. 48:129).

In addition to cortisol, the biological effects of other steroids can be modulated at the GR level using receptor modulators, such as agonists, partial agonists and antagonists. When administered to subjects in need thereof, steroids can provide both intended therapeutic effects, e.g., by stimulating glucocorticoid receptor transrepression, as well as negative side effects, e.g. by chronic glucocorticoid receptor transactivation. Miricorilant (CORT118335) is another such glucocorticoid receptor modulator compound, and has been described previously in PCT Publication No. WO 2012/129074, and U.S. Pat. No. 8,685,973. What is needed in the art are new methods of preparing miricorilant having lower impurity content. Surprisingly, the present invention meets these and other needs.

### BRIEF SUMMARY OF THE INVENTION

In one embodiment, the present invention provides a method of preparing a compound of Formula I:

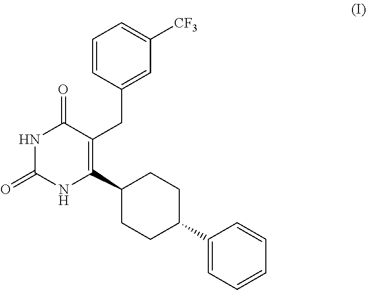 [SMILES: O=c1[nH]c(=O)c(Cc2cccc(C(F)(F)F)c2)c([C@H]2CC[C@H](c3ccccc3)CC2)[nH]1]

the method comprising:
(a) forming a first reaction mixture comprising a first solvent, a compound of Formula VIII:

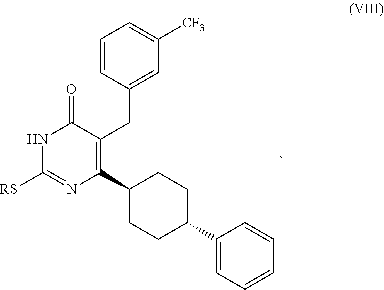

and an oxidizing agent, under conditions suitable to prepare the compound of Formula I,
wherein R is C₁₋₁₂ alkyl.

In another embodiment, the present invention provides a method of preparing a compound of Formula VIII:

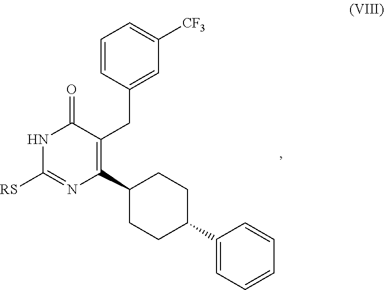

the method comprising:
(b) forming a second reaction mixture comprising an alkylating agent, a second non-nucleophilic base, a second solvent, and a compound of Formula IX:

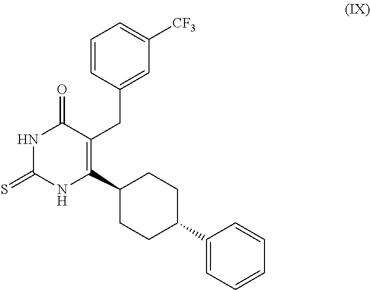 [SMILES: O=c1[nH]c(=S)[nH]c([C@H]2CC[C@H](c3ccccc3)CC2)c1Cc1cccc(C(F)(F)F)c1]

under conditions suitable to form the compound of Formula VIII,
wherein R is C₁₋₁₂ alkyl;
the alkylating agent is C₁₋₁₂ alkyl-halide or C₁₋₁₂ alkyl-OS(O)2R^{a};
R^{a} is C₁₋₆ alkyl, C₁₋₆ haloalkyl, and phenyl substituted with 0, 1, 2 or 3 R^{a1}; and
each R^{a1} is independently C₁₋₆ alkyl, halogen, or C₁₋₆ haloalkyl.

In another embodiment, the present invention provides a method of preparing a compound of Formula IX:

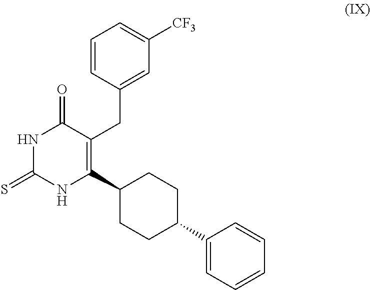 [SMILES: O=c1[nH]c(=S)[nH]c([C@H]2CC[C@H](c3ccccc3)CC2)c1Cc1cccc(C(F)(F)F)c1]

the method comprising:
(c) forming a third reaction mixture comprising a thiourea, a third non-nucleophilic base, a third solvent, and a compound of Formula III:

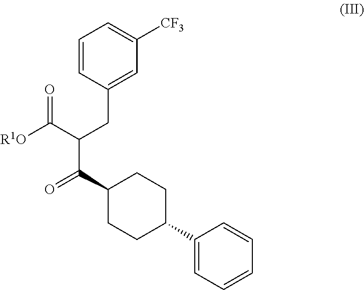

under conditions suitable to prepare the compound of Formula IX,
wherein R¹ is C₁₋₆ alkyl.

In another embodiment, the present invention provides a compound of Formula VIII:

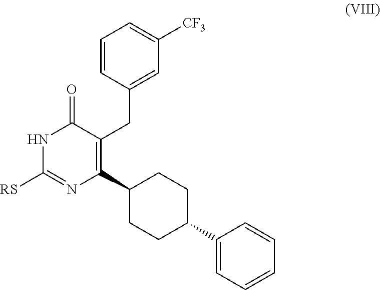

wherein R is C₁₋₁₂ alkyl.

### BRIEF SUMMARY OF THE DRAWINGS

FIG. 1 shows the proton NMR for the compound of Formula VIII.

FIG. 2 shows the XRPD for the compound of Formula VIII.

### DETAILED DESCRIPTION OF THE INVENTION

### I. General

The present disclosure describes methods of preparing 6-((1r,4r)-4-phenylcyclohexyl)-5-(3-(trifluoromethyl)benzyl)pyrimidine-2,4 (1H,3H)-dione (Formula I), Example 6 of U.S. Pat. No. 8,685,973 via a thioether intermediate prepared from the sulfone of Formula IX. The present disclosure also describes new intermediates.

### II. Definitions

“About” when referring to a value includes the stated value +/−10% of the stated value. For example, about 50% includes a range of from 45% to 55%, while about 10 equivalents includes a range of from 9 to 11 equivalents. Accordingly, when referring to a range, “about” refers to each of the stated values +/−10% of the stated value of each end of the range. For instance, a ratio of from about 1 to about 10 (w/w) includes a range of from 0.9 to 11.

“Forming a reaction mixture” refers to the process of bringing into contact at least two distinct species such that they mix together and can react. It should be appreciated, however, the resulting reaction product can be produced directly from a reaction between the added reagents or from an intermediate from one or more of the added reagents which can be produced in the reaction mixture.

“Solvent” refers to a substance, such as a liquid, capable of dissolving a solute. Solvents can be polar or non-polar, protic or aprotic. Polar solvents typically have a dielectric constant greater than about 5 or a dipole moment above about 1.0, and non-polar solvents have a dielectric constant below about 5 or a dipole moment below about 1.0. Protic solvents are characterized by having a proton available for removal, such as by having a hydroxy or carboxy group. Aprotic solvents lack such a group. Representative polar protic solvents include alcohols (methanol, ethanol, propanol, isopropanol, etc.), acids (formic acid, acetic acid, etc.) and water. Representative polar aprotic solvents include dichloromethane, chloroform, 1,4-dioxane, tetrahydrofuran, diethyl ether, acetone, ethyl acetate, N,N-dimethylformamide, dimethylacetamide, acetonitrile and dimethyl sulfoxide. Representative non-polar solvents include alkanes (pentanes, hexanes, etc.), cycloalkanes (cyclopentane, cyclohexane, etc.), benzene, and toluene. Other solvents are useful in the present invention.

“Acid” refers to a compound capable of donating a proton (a Bronsted-Lowry acid) or capable of accepting an electron pair (a Lewis acid). Representative acids include, but are not limited to, hydrochloric acid, sulfuric acid, formic acid, acetic acid, propanoic acid, butyric acid, hexanoic acid, octanoic acid, trifluoroacetic acid, tetrafluoroboric acid (HBF₄), etc.

“Strong acid” refers to an acid that easily dissociates, which is often represented by a pKₐ less than −1 in water. Representative strong acids include, but are not limited to, hydrochloric acid, hydrobromic acid, hydroiodic acid, nitric acid, sulfuric acid, and perchloric acid.

“Hydrate” refers to a compound that is complexed to at least one water molecule. The compounds of the present invention can be complexed with from 1 to 10 water molecules.

“Oxidizing agent” or “oxidizer” refers to a reagent capable of accepting an electron pair from another compound, thus oxidizing the compound. Representative oxidizing agents include, but are not limited to, oxygen, hydrogen peroxide, nitrite, nitric acid, sulfuric acids, etc.

“Alkyl” refers to a straight or branched acyclic hydrocarbon containing normal, secondary, or tertiary carbon atoms. For example, an alkyl group can have 1 to 20 carbon atoms (i.e, C₁-C₂₀ alkyl), 1 to 12 carbon atoms (i.e., C₁-C₁₂ alkyl), or 1 to 6 carbon atoms (i.e., C₁-C₆ alkyl). Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₄, C₂₋₅, C₂₋₆, C₃₋₄, C₃₋₅, C₃₋₆, C₄₋₅, C₄₋₆ and C₅₋₆. Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (s-Pn, s-Pentyl, —CH(CH₃)CH₂CH₂CH₃), 3-pentyl (—CH(CH₂CH₃)₂), 2-methyl-2-butyl (t-Pn, t-Pentyl, —C(CH₃)₂CH₂CH₃), 3-methyl-2-butyl (neo-Pn, neo-Pentyl, —CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃) (CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃).

“Halogen” refers to fluorine, chlorine, bromine and iodine.

“Alkylating agent” refers to a reagent having both an alkyl group and a leaving group and is capable of reacting with a second reagent to install the alkyl group on the second reagent. Representative alkylating agents include, but are not limited to, alkyl-halides, alkyl-sulfonates, etc.

“Alkyl-halide” refers to an alkyl group linked to a single halogen. Representative alkyl-halides include, but are not limited to, n-octyl-iodide.

“Alkylating additive” refers to an additive that converts an alkyl chloride or alkyl bromide to an alkyl iodide. Representative alkylating additives include, but are not limited to, sodium bromide, sodium iodide, potassium bromide, potassium iodide, or tetraalkylammonium bromide, iodide salts such as those commonly used as phase transfer catalysts, and combinations thereof.

“Non-nucleophilic base” refers to a compound capable of accepting a proton (H⁺) under the Bronsted-Lowry definition, or is an electron-pair donor under the Lewis definition, that is not nucleophilic. Non-nucleophilic bases useful in the present invention include amines such as trimethylamine, triethylamine, N,N-diisopropylethylamine (DIPEA or Hunig's Base), 1,8-diazabicycloundec-7-ene (DBU), 2,6-di-tert-butylpyridine, quinuclidine, and lithium di-isopropylamine (LDA). Other bases are known to one of skill in the art.

“Thiourea” refers to H₂NC(═S)NH₂.

“Cooling” refers to applying cooling means to the reaction mixture to decrease the temperature of the reaction mixture by at least 1 degree Celsius. For example, cooling can include, but is not limited to, decreasing the temperature of the reaction mixture to or below room temperature.

“Heating” refers to applying heat to the reaction mixture to increase the temperature of the reaction mixture by at least 1 degree Celsius. For example, heating can include, but is not limited to, raising the temperature of the reaction mixture to room temperature, or to the reflux or boiling temperature of the reaction mixture, or to a temperature between room temperature and the reflux or boiling temperature of the reaction mixture.

“Room temperature” is the range of air temperatures generally considered to be suitable for human occupancy, or between about 15 degrees Celsius (59 degrees Fahrenheit) and 25 degrees Celsius (77 degrees Fahrenheit).

“Crystalline seed” refers to a seed crystal of the target crystalline form to be prepared.

### III. Sulfone Method of Preparing Formula I

The present invention provides methods for the preparation of 6-((1r,4r)-4-phenylcyclohexyl)-5-(3-(trifluoromethyl)benzyl)pyrimidine-2,4 (1H,3H)-dione, the compound of Formula I:

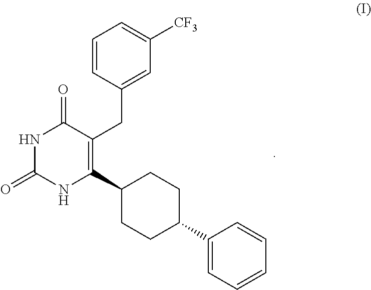 [SMILES: O=c1[nH]c(=O)c(Cc2cccc(C(F)(F)F)c2)c([C@H]2CC[C@H](c3ccccc3)CC2)[nH]1]

The compound of Formula I was originally disclosed as Example 6 in U.S. Pat. No. 8,685,973.

A. Preparation of Formula I from Formula VIII

In some embodiments, the present invention provides a method of preparing a compound of Formula I:

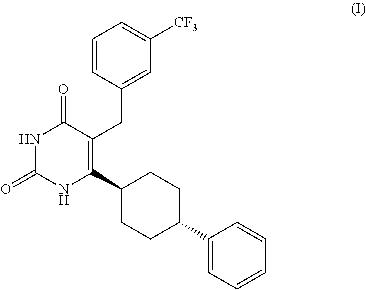 [SMILES: O=c1[nH]c(=O)c(Cc2cccc(C(F)(F)F)c2)c([C@H]2CC[C@H](c3ccccc3)CC2)[nH]1]

the method comprising: forming a first reaction mixture comprising a first solvent, a compound of Formula VIII:

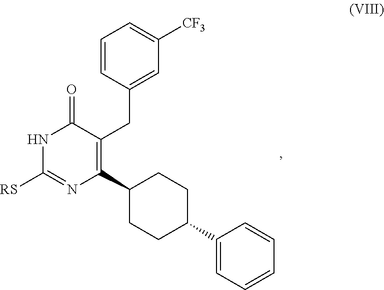

and an oxidizing agent, under conditions suitable to prepare the compound of Formula I, wherein R is C₁₋₁₂ alkyl.

Any suitable solvent can be used as the first solvent in the first reaction mixture of the present invention. Representative solvents include, but are not limited to, polar protic solvents, polar aprotic solvents, and non-polar solvents. In some embodiments, the method of preparing the compound of Formula I includes the method wherein the first solvent comprises acetone, methyl acetate, ethyl acetate, isopropyl acetate, N,N-dimethylformamide (DMF), N,N-dimethylacetamide (DMAc), dimethylsulfoxide (DMSO), 2-methyltetrahydrofuran (2-MeTHF), tetrahydrofuran (THF), N-methyl-2-pyrrolidone (NMP), or combinations thereof. In some embodiments, the method of preparing the compound of Formula I includes the method wherein the first solvent comprises N-methyl-2-pyrrolidone (NMP).

In some embodiments, the method of preparing the compound of Formula I includes the method wherein R is C₆₋₁₂ alkyl. In some embodiments, the method of preparing the compound of Formula I includes the method wherein R is n-hexyl, n-heptyl, n-octyl, n-nonyl, or n-decyl. In some embodiments, the method of preparing the compound of Formula I includes the method wherein R is n-octyl.

In some embodiments, the method of preparing the compound of Formula I includes the method wherein the compound of Formula VIII has the structure:

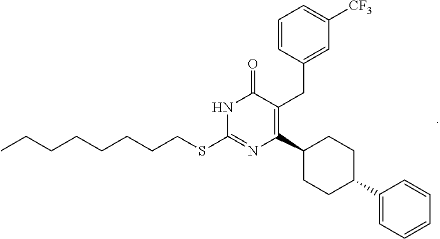 [SMILES: CCCCCCCCSc1nc([C@H]2CC[C@H](c3ccccc3)CC2)c(Cc2cccc(C(F)(F)F)c2)c(=O)[nH]1]

Any suitable oxidizing agent can be used as the oxidizing agent in the first reaction mixture of the present invention. Representative oxidizing agents include, but are not limited to, potassium peroxymonosulfate (OXONE®), hydrogen peroxide, nitric acid, potassium chlorate, sulfuric acid, peroxydisulfuric acid, hypochlorite, and potassium permanganate. In some embodiments, the method of preparing the compound of Formula I includes the method wherein the oxidizing agent comprises potassium peroxymonosulfate (oxone), hydrogen peroxide, nitric acid, potassium chlorate, sulfuric acid, peroxydisulfuric acid, hypochlorite, potassium permanganate, or combinations thereof.

In some embodiments, the method of preparing the compound of Formula I includes the method wherein the oxidizing agent comprises potassium peroxymonosulfate (OXONE®). In some embodiments, the method of preparing the compound of Formula I includes the method (a) forming the first reaction mixture comprising potassium peroxomonosulfate (OXONE®), N-methyl-2-pyrrolidone, and the compound of Formula VIII, under conditions suitable to prepare the compound of Formula I.

In some embodiments, the method of preparing the compound of Formula I includes the method wherein the oxidizing agent comprises hydrogen peroxide. In some embodiments, the method of preparing the compound of Formula I includes the method (a) forming the first reaction mixture comprising hydrogen peroxide, N-methyl-2-pyrrolidone, and the compound of Formula VIII, under conditions suitable to prepare the compound of Formula I.

The oxidizing agent can be present in any suitable ratio to the compound of Formula VIII. For example, the oxidizing agent can be present in an amount of from 0.1 to 10 molar equivalents to the compound of Formula VIII, or from 1 to 10, from 1 to 5, from 1 to 7, from 4 to 6, or from 4.5 to 5.5 molar equivalents to the compound of Formula VIII. Representative amounts of the oxidizing agent include, but are not limited to, about 1.0 molar equivalent to the compound of Formula VIII, or about 1.5, 2.0, 2.5, 3.0, 3.5, 4.0, 4.5, 4.6, 4.7, 4.8, 4.9, 5.0, 5.1, 5.2, 5.3, 5.4, 5.5, 6.0, 6.5, 7.0, 7.5, 8.0, 8.5, 9.0, 9.5, or about 10 molar equivalents to the compound of Formula VIII.

In some embodiments, the method of preparing the compound of Formula I includes the method wherein the oxone is present in an amount of from 3 to 7 molar equivalents to the compound of Formula VIII. In some embodiments, the method of preparing the compound of Formula I includes the method wherein the oxone is present in an amount of about 5 molar equivalents to the compound of Formula VIII.

In some embodiments, the method of preparing the compound of Formula I includes the method wherein the hydrogen peroxide is present in an amount of from 1 to 10 molar equivalents to the compound of Formula VIII. In some embodiments, the method of preparing the compound of Formula I includes the method wherein the hydrogen peroxide is present in an amount of from 3 to 7 molar equivalents to the compound of Formula VIII.

In some embodiments, the method of preparing the compound of Formula I includes the method wherein the first reaction mixture further comprises a strong acid. Any strong acid can be used as the strong acid in the first reaction mixture of the present invention. Representative strong acids include, but are not limited to, organic acids and inorganic acids. In some embodiments, the method of preparing the compound of Formula I includes the method wherein the strong acid comprises trifluoroacetic acid, trichloroacetic acid, ethane-1,2-disulfonic acid, naphthalene-2-sulfonic acid, naphthalene-1,5-disulfonic acid, hydrofluoric acid, hydrochloric acid, hydrobromic acid, hypochlorous acid, chloric acid, perchloric acid, sulfuric acid, nitric acid, phosphoric acid, methanesulfonic acid, ethanesulfonic acid, benzenesulfonic acid, p-toluenesulfonic acid, trifluoromethanesulfonic acid, camphorsulfonic acid, or combinations thereof. In some embodiments, the method of preparing the compound of Formula I includes the method wherein the strong acid comprises sulfuric acid.

The strong acid can be present in any suitable ratio to the compound of Formula VIII. For example, the strong acid can be present in an amount of from 0.1 to 10 molar equivalents to the compound of Formula VIII, or from 1 to 10, from 1 to 5, from 2 to 4, or from 2.5 to 3.5 molar equivalents to the compound of Formula VIII. Representative amounts of the strong acid include, but are not limited to, about 1.0 molar equivalent to the compound of Formula VIII, or about 1.5, 2.0, 2.5, 3.0, 3.5, 4.0, 4.5, 5.0, 5.5, 6.0, 6.5, 7.0, 7.5, 8.0, 8.5, 9.0, 9.5, or about 10 molar equivalents to the compound of Formula VIII.

In some embodiments, the method of preparing the compound of Formula I includes the method wherein the sulfuric acid is present in an amount of from 1 to 5 molar equivalents to the compound of Formula VIII. In some embodiments, the method of preparing the compound of Formula I includes the method wherein the sulfuric acid is present in an amount of about 3 molar equivalents to the compound of Formula VIII.

In some embodiments, the method of preparing the compound of Formula I includes the method (a) forming the first reaction mixture comprising oxone in an amount of about 5 molar equivalents to the compound of Formula VIII, N-methyl-2-pyrrolidone, and the compound of Formula VIII, under conditions suitable to prepare the compound of Formula I.

In some embodiments, the method of preparing the compound of Formula I includes the method (a) forming the first reaction mixture comprising hydrogen peroxide in an amount of from 3 to 7 molar equivalents to the compound of Formula VIII, sulfuric acid, N-methyl-2-pyrrolidone, and the compound of Formula VIII, under conditions suitable to prepare the compound of Formula I. In some embodiments, the method of preparing the compound of Formula I includes the method (a) forming the first reaction mixture comprising hydrogen peroxide in an amount of 3 to 7 molar equivalents to the compound of Formula VIII, sulfuric acid in an amount of about 3 molar equivalents to the compound of Formula VIII, N-methyl-2-pyrrolidone, and the compound of Formula VIII, under conditions suitable to prepare the compound of Formula I.

B. Preparation of Formula VIII from Formula IX

In some embodiments, the present invention provides a method of preparing a compound of Formula VIII:

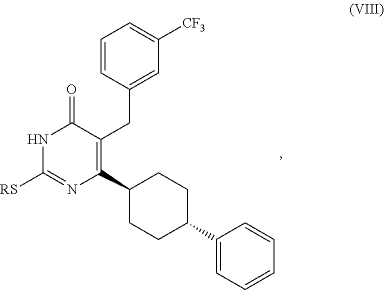

the method comprising: (b) forming a second reaction mixture comprising an alkylating agent, a second non-nucleophilic base, a second solvent, and a compound of Formula IX:

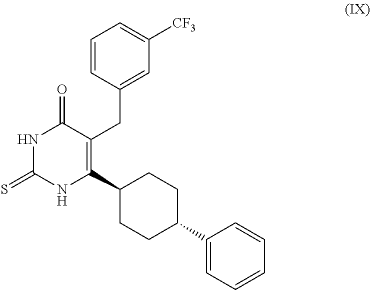 [SMILES: O=c1[nH]c(=S)[nH]c([C@H]2CC[C@H](c3ccccc3)CC2)c1Cc1cccc(C(F)(F)F)c1]

under conditions suitable to form the compound of Formula VIII, wherein R is C₁₋₁₂ alkyl; the alkylating agent is C₁₋₁₂ alkyl-halide or C₁₋₁₂ alkyl-OS(O)2R^{a}; R^{a} is C₁₋₆ alkyl, C₁₋₆ haloalkyl, and phenyl substituted with 0, 1, 2 or 3 R^{a1}; and each R^{a1} is independently C₁₋₆ alkyl, halogen, or C₁₋₆ haloalkyl.

Any suitable alkylating agent can be used in the method of preparing Formula VIII. For example, the alkylating agent can be an alkyl halide, alkyl sulfonate, or other groups. In some embodiments, the alkylating agent is C₁₋₁₂ alkyl-halide. In some embodiments, the alkylating agent is C₁₋₁₂ alkyl-OS(O)2R^{a}. In some embodiments, the alkylating agent is C₆₋₁₂ alkyl-OS(O)2R^{a}, wherein R^{a} is methyl, trifluoromethyl, or 4-methyl-phenyl. In some embodiments, the alkylating agent is C₁₋₁₂ alkyl-mesylate, C₁₋₁₂ alkyl-triflate, or C₁₋₁₂ alkyl-tosylate.

Any suitable alkyl halide can be used as the C₁₋₁₂ alkyl halide in the second reaction mixture. Representative C₁₋₁₂ alkyl halides include, but are not limited to, alkyl iodides, alkyl bromides, and alkyl chlorides. In some embodiments, the method of preparing the compound of Formula VIII includes the method wherein the second reaction mixture comprises a C₆₋₁₂ alkyl-iodide. In some embodiments, the method of preparing the compound of Formula VIII includes the method wherein the second reaction mixture comprises n-hexyl-iodide, n-heptyl-iodide, n-octyl-iodide, n-nonyl-iodide, or n-decyl-iodide. In some embodiments, the method of preparing the compound of Formula VIII includes the method wherein the second reaction mixture comprises n-octyl-iodide.

The alkylating agent or C₁₋₁₂ alkyl halide can be present in any suitable amount to the compound of Formula IX. For example, the alkylating agent or C₁₋₁₂ alkyl halide can be present in an amount of from 0.1 to 10 molar equivalents to the compound of Formula IX, or from 0.2 to 5, from 0.5 to 3, from 0.8 to 3, from 0.9 to 1.5, or from 0.9 to 1.1 molar equivalents to the compound of Formula IX. Representative amounts of the alkylating agent or C₁₋₁₂ alkyl halide include, but are not limited to, about 0.5 molar equivalents to the compound of Formula IX, or about 0.6, 0.7, 0.8, 0.9, 1.0, 1.1, 1.2, 1.3, 1.4, 1.5, 2.0, 2.5, 3.0, 4.0, 5.0, 6.0, 7.0, 8.0, 9.0, or 10 molar equivalents to the compound of Formula IX. In some embodiments, the method of preparing the compound of Formula VIII includes the method wherein the alkyl-iodide is present in an amount of 0.9 to 2 molar equivalents to the compound of Formula IX. In some embodiments, the method of preparing the compound of Formula VIII includes the method wherein the alkyl-iodide is present in an amount of about 1 molar equivalent to the compound of Formula IX.

The alkyl iodide alkylating agent can also be prepared in situ from an alkyl chloride or alkyl bromide with an additive such as, but not limited to, sodium bromide, sodium iodide, potassium bromide, potassium iodide, or tetraalkylammonium bromide, iodide salts such as those commonly used as phase transfer catalysts, and combinations thereof. In some embodiments, the alkylating agent is an alkyl chloride or alkyl bromide, and the second reaction mixture further comprises an alkylating additive, such that an alkyl iodide is formed in situ. In some embodiments, the alkylating additive can be sodium bromide, sodium iodide, potassium bromide, potassium iodide, or tetraalkylammonium bromide, iodide salt, or combinations thereof.

In some embodiments, the method of preparing the compound of Formula VIII includes the method comprising: (b) forming the second reaction mixture comprising alkyl bromide, the alkylating additive, potassium carbonate, N-methyl-2-pyrrolidone, and the compound of Formula IX, under conditions suitable to form the compound of Formula VIII. In some embodiments, the method of preparing the compound of Formula VIII includes the method comprising: (b) forming the second reaction mixture comprising alkyl chloride, the alkylating additive, potassium carbonate, N-methyl-2-pyrrolidone, and the compound of Formula IX, under conditions suitable to form the compound of Formula VIII.

The alkylating additive can be present in the second reaction mixture in any suitable amount to the compound of Formula IX. For example, the alkylating additive can be present in a catalytic amount, or an amount of from 0.01 to 10 molar equivalents to the compound of Formula IX, or from 0.05 to 5, or from 0.1 to 1 molar equivalents to the compound of Formula IX.

Any non-nucleophilic base can be used as the second non-nucleophilic base in the second reaction mixture of the present invention. Representative second non-nucleophilic bases include, but are not limited to, potassium carbonate, sodium carbonate, alkoxides such as potassium tert-butoxide and sodium tert-butoxide, hexamethylsilazane (HMDS), lithium hexamethyldisilazane, sodium hexamethyldisilazine, potassium hexamethyldisilazane, lithium diisopropylamine (LDA), lithium hydride, sodium hydride, potassium hydride, n-butyl lithium, as well as amine bases, such as triethylamine (Et₃N), N,N-diisopropylethylamine (iPr₂NEt; DIPEA), 1,8-diazabicycloundec-7-ene (DBU), 1,5-diazabicyclo(4.3.0) non-5-ene (DBN), 1,4-diazabicyclo[2.2.2]octane (DABCO), N,N-diethylaniline, pyridine, 2,6-lutidine, 2,4,6-collidine, 4-dimethylaminopyridine, and quinuclidine. Non-nucleophilic base includes non-nucleophilic amine bases.

In some embodiments, the method of preparing the compound of Formula VIII includes the method wherein the second non-nucleophilic base comprises sodium carbonate, potassium carbonate, cesium carbonate, triethylamine, N,N-diisopropyl ethylamine (DIPEA), N,N-dimethyl isopropylamine (DIMPA), piperidine, 1-ethylpiperidine, N-methylmorpholine, N-methylpyrrolidine, N,N-dimethylamine, piperazine, N-methylpiperazine, pyridine, N,N-dimethylaniline, N,N-diethylaniline, 2,6-lutidine, 2,4,6-collidine, 4-dimethyl aminopyridine (DMAP), quinuclidine, 4-pyrrolidinopyridine, 1,5-diazabicyclo(4.3.0) non-5-ene (DBN), 1,8-diazabicyclo[5.4.0]undec-7-ene (DBU), 1,4-diazabicyclo[2.2.2]octane (DABCO), or combinations thereof. In some embodiments, the method of preparing the compound of Formula VIII includes the method wherein the second non-nucleophilic base comprises sodium carbonate, potassium carbonate, or cesium carbonate. In some embodiments, the method of preparing the compound of Formula VIII includes the method wherein the second non-nucleophilic base comprises potassium carbonate.

The second non-nucleophilic base can be present in any suitable amount to the compound of Formula IX. For example, the second non-nucleophilic base can be present in an amount of from 0.1 to 10 molar equivalents to the compound of Formula IX, or from 0.2 to 5, from 0.5 to 3, from 0.8 to 2, from 0.9 to 1.5, or from 0.9 to 1.1 molar equivalents to the compound of Formula IX. Representative amounts of the second non-nucleophilic base include, but are not limited to, about 0.5 molar equivalents to the compound of Formula IX, or about 0.6, 0.7, 0.8, 0.9, 1.0, 1.1, 1.2, 1.3, 1.4, 1.5, 2.0, 2.5, 3.0, 4.0, 5.0, 6.0, 7.0, 8.0, 9.0, or 10 molar equivalents to the compound of Formula IX. In some embodiments, the method of preparing the compound of Formula VIII includes the method wherein the second non-nucleophilic base is present in an amount of from 0.9 to 2 molar equivalents to the compound of Formula IX. In some embodiments, the method of preparing the compound of Formula VIII includes the method wherein the second non-nucleophilic base is present in an amount of about 1 molar equivalent to the compound of Formula IX.

Any suitable solvent can be used as the second solvent in the second reaction mixture of the present invention. Representative second solvents include, but are not limited to, polar protic solvents, polar aprotic solvents, and non-polar solvents. In some embodiments, the method of preparing the compound of Formula VIII includes the method wherein the second solvent comprises acetone, methyl acetate, ethyl acetate, isopropyl acetate, N,N-dimethylformamide (DMF), N,N-dimethylacetamide (DMAc), dimethylsulfoxide (DMSO), 2-methyltetrahydrofuran (2-MeTHF), tetrahydrofuran (THF), N-methyl-2-pyrrolidone (NMP), or combinations thereof. In some embodiments, the method of preparing the compound of Formula VIII includes the method wherein the second solvent comprises N,N-dimethylformamide (DMF), N,N-dimethylacetamide (DMAc), N-methyl-2-pyrrolidone (NMP), or combinations thereof. In some embodiments, the method of preparing the compound of Formula VIII includes the method wherein the second solvent comprises N-methyl-2-pyrrolidone (NMP).

In some embodiments, the method of preparing the compound of Formula VIII includes the method comprising: (b) forming the second reaction mixture comprising n-octyl-iodide in an amount of about 1 molar equivalent to the compound of Formula IX, potassium carbonate in an amount of about 1 molar equivalent to the compound of Formula IX, N-methyl-2-pyrrolidone, and the compound of Formula IX, under conditions suitable to form the compound of Formula VIII.

In some embodiments, the method of preparing the compound of Formula VIII includes the method wherein the compound of Formula VIII is prepared by the methods of the present invention.

In some embodiments, the method of preparing the compound of Formula VIII includes the method comprising: (b) forming the second reaction mixture comprising n-octyl-iodide in an amount of about 1 molar equivalent to the compound of Formula IX, potassium carbonate in an amount of about 1 molar equivalent to the compound of Formula IX, N-methyl-2-pyrrolidone, and the compound of Formula IX, under conditions suitable to form the compound of Formula VIII; and (a) forming the first reaction mixture comprising hydrogen peroxide in an amount of from 3 to 7 molar equivalents to the compound of Formula VIII, sulfuric acid, N-methyl-2-pyrrolidone, and the compound of Formula VIII, under conditions suitable to prepare the compound of Formula I.

C. Preparation of Formula IX from Formula III

The present invention provides a method of preparing a compound of Formula IX:

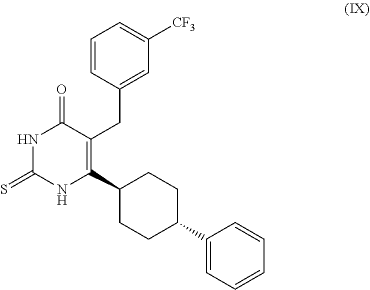 [SMILES: O=c1[nH]c(=S)[nH]c([C@H]2CC[C@H](c3ccccc3)CC2)c1Cc1cccc(C(F)(F)F)c1]

the method comprising:
(c) forming a third reaction mixture comprising a thiourea, a third non-nucleophilic base, a third solvent, and a compound of Formula III:

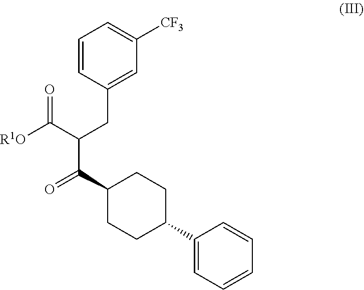

under conditions suitable to prepare the compound of Formula IX, wherein R¹ is C₁₋₆ alkyl.

In some embodiments, the method of preparing the compound of Formula IX is the method wherein R¹ is methyl, ethyl, n-propyl, isopropyl, n-butyl, sec-butyl, tert-butyl, isobutyl, pentyl, or hexyl. In some embodiments, the method of preparing the compound of Formula IX is the method wherein R¹ is methyl, ethyl or n-propyl. In some embodiments, the method of preparing the compound of Formula IX is the method wherein R¹ is ethyl.

In some embodiments, the method of preparing the compound of Formula IX is the method wherein the compound of Formula III has the structure:

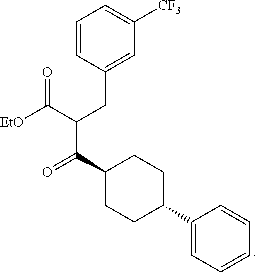 [SMILES: CCOC(=O)C(Cc1cccc(C(F)(F)F)c1)C(=O)[C@H]1CC[C@H](c2ccccc2)CC1 ; "."]

Any suitable solvent can be the third solvent in the third reaction mixture of the present invention. Representative third solvents include, but are not limited to, polar protic solvents, polar aprotic solvents, and non-polar solvents. In some embodiments, the method of preparing the compound of Formula IX is the method wherein the third solvent comprises acetone, methyl acetate, ethyl acetate, isopropyl acetate, N,N-dimethylformamide (DMF), N,N-dimethylacetamide (DMAc), dimethylsulfoxide (DMSO), 2-methyltetrahydrofuran (2-MeTHF), tetrahydrofuran (THF), N-methyl-2-pyrrolidone (NMP), or combinations thereof. In some embodiments, the method of preparing the compound of Formula IX is the method wherein the third solvent comprises N,N-dimethylformamide (DMF), N,N-dimethylacetamide (DMAc), N-methyl-2-pyrrolidone (NMP), or combinations thereof. In some embodiments, the method of preparing the compound of Formula IX is the method wherein the third solvent comprises N,N-dimethylformamide (DMF).

Any non-nucleophilic base can be used as the third non-nucleophilic base in the third reaction mixture of the present invention. Representative third non-nucleophilic bases include, but are not limited to, potassium carbonate, sodium carbonate, alkoxides such as potassium tert-butoxide and sodium tert-butoxide, hexamethylsilazane (HMDS), lithium hexamethyldisilazane, sodium hexamethyldisilazine, potassium hexamethyldisilazane, lithium diisopropylamine (LDA), lithium hydride, sodium hydride, potassium hydride, n-butyl lithium, as well as amine bases, such as triethylamine (Et₃N), N,N-diisopropylethylamine (iPr₂NEt; DIPEA), 1,8-diazabicycloundec-7-ene (DBU), 1,5-diazabicyclo(4.3.0) non-5-ene (DBN), 1,4-diazabicyclo[2.2.2]octane (DABCO), N,N-diethylaniline, pyridine, 2,6-lutidine, 2,4,6-collidine, 4-dimethylaminopyridine, and quinuclidine. Non-nucleophilic base includes non-nucleophilic amine bases.

In some embodiments, the method of preparing the compound of Formula IX is the method wherein the third non-nucleophilic base comprises triethylamine, N,N-diisopropyl ethylamine (DIPEA), N,N-dimethyl isopropylamine (DIMPA), piperidine, 1-ethylpiperidine, N-methylmorpholine, N-methylpyrrolidine, N,N-dimethylamine, piperazine, N-methylpiperazine, pyridine, N,N-dimethylaniline, N,N-diethylaniline, 2,6-lutidine, 2,4,6-collidine, 4-dimethyl aminopyridine (DMAP), quinuclidine, 4-pyrrolidinopyridine, 1,5-diazabicyclo(4.3.0) non-5-ene (DBN), 1,8-diazabicyclo[5.4.0]undec-7-ene (DBU), 1,4-diazabicyclo[2.2.2]octane (DABCO), or combinations thereof. In some embodiments, the method of preparing the compound of Formula IX is the method wherein the third non-nucleophilic base comprises 1,8-diazabicyclo[5.4.0]undec-7-ene (DBU).

The third non-nucleophilic base can be present in any suitable amount to the compound of Formula III. For example, the third non-nucleophilic base can be present in an amount of from 1 to 10 molar equivalents to the compound of Formula III, or from 1 to 8, from 1.5 to 7, from 2 to 6, from 2 to 3, from 2.1 to 2.9, from 2.2 to 2.8, from 2.3 to 2.7, or from 2.4 to 2.6 molar equivalents to the compound of Formula III. Representative amounts of the third non-nucleophilic base include, but are not limited to, about 0.5 molar equivalents to the compound of Formula III, or about 1.0, 1.5, 2.0, 2.1, 2.2, 2.3, 2.4, 2.5, 2.6, 2.7, 2.8, 2.9, 3.0, 3.5, 4.0, 5.0, 6.0, 7.0, 8.0, 9.0, or about 10 molar equivalents to the compound of Formula III. In some embodiments, the method of preparing the compound of Formula IX is the method wherein the third non-nucleophilic base is present in an amount of from 1 to 5 molar equivalents to the compound of Formula III. In some embodiments, the method of preparing the compound of Formula IX is the method wherein the third non-nucleophilic base is present in an amount of about 2.5 molar equivalents to the compound of Formula III.

The thiourea can be present in any suitable amount to the compound of Formula III. For example, the thiourea can be present in an amount of from 1 to 10 molar equivalents to the compound of Formula III, or from 1 to 8, from 2 to 7, from 5 to 7, from 5.5 to 6.5, from 5.6 to 6.4, from 5.7 to 6.3, from 5.8 to 6.2, or from 5.9 to 6.1 molar equivalents to the compound of Formula III. Representative amounts of the thiourea include, but are not limited to, about 1.0 molar equivalent to the compound of Formula III, or about 2.0, 3.0, 4.0, 4.5, 5.0, 5.5, 5.6, 5.7, 5.8, 5.9, 6.0, 6.1, 6.2, 6.3, 6.4, 6.5, 7.0, 8.0, 9.0, or about 10 molar equivalents to the compound of Formula III.

In some embodiments, the method of preparing the compound of Formula IX includes the method wherein the thiourea is present in an amount from 5 to 7 molar equivalents to the compound of Formula III. In some embodiments, the method of preparing the compound of Formula IX includes the method wherein the thiourea is present in an amount of about 6 molar equivalents to the compound of Formula III.

In some embodiments, the method of preparing the compound of Formula IX is the method comprising (c) forming the third reaction mixture comprising thiourea, DBU in an amount of about 2.5 molar equivalents to the compound of Formula III, N,N-dimethylformamide, and the compound of Formula III, under conditions suitable to prepare the compound of Formula IX.

In some embodiments, the method of preparing the compound of Formula IX is the method comprising (c) forming the third reaction mixture comprising thiourea in an amount of about 6 molar equivalents to the compound of Formula III, DBU in an amount of about 2.5 molar equivalents to the compound of Formula III, N,N-dimethylformamide, and the compound of Formula III, under conditions suitable to prepare the compound of Formula IX.

In some embodiments, the method of preparing the compound of Formula VIII is the method wherein the compound of Formula IX is prepared by the methods of the present invention. In some embodiments, the method of preparing the compound of Formula I is the method wherein the compound of Formula IX is prepared by the methods of the present invention.

In some embodiments, the method of preparing the compound of Formula VIII or the method of preparing the compound of Formula I is the method comprising: (c) forming the third reaction mixture comprising thiourea, DBU in an amount of about 2.5 molar equivalents to the compound of Formula III, N,N-dimethylformamide, and the compound of Formula III, under conditions suitable to prepare the compound of Formula IX; (b) forming the second reaction mixture comprising n-octyl-iodide in an amount of about 1 molar equivalent to the compound of Formula IX, potassium carbonate in an amount of about 1 molar equivalent to the compound of Formula IX, N-methyl-2-pyrrolidone, and the compound of Formula IX, under conditions suitable to form the compound of Formula VIII; and (a) forming the first reaction mixture comprising potassium peroxomonosulfate (oxone), N-methyl-2-pyrrolidone, and the compound of Formula VIII, under conditions suitable to prepare the compound of Formula I.

In some embodiments, the method of preparing the compound of Formula VIII or the method of preparing the compound of Formula I is the method comprising: (c) forming the third reaction mixture comprising thiourea, DBU in an amount of about 2.5 molar equivalents to the compound of Formula III, N,N-dimethylformamide, and the compound of Formula III, under conditions suitable to prepare the compound of Formula IX; (b) forming the second reaction mixture comprising n-octyl-iodide in an amount of about 1 molar equivalent to the compound of Formula IX, potassium carbonate in an amount of about 1 molar equivalent to the compound of Formula IX, N-methyl-2-pyrrolidone, and the compound of Formula IX, under conditions suitable to form the compound of Formula VIII; and (a) forming the first reaction mixture comprising hydrogen peroxide in an amount of from 3 to 7 molar equivalents to the compound of Formula VIII, sulfuric acid, N-methyl-2-pyrrolidone, and the compound of Formula VIII, under conditions suitable to prepare the compound of Formula I.

### D. Sulfone Intermediate Compounds

The present invention provides compounds of Formula VIII. In some embodiments, the present invention provides a compound of Formula VIII:

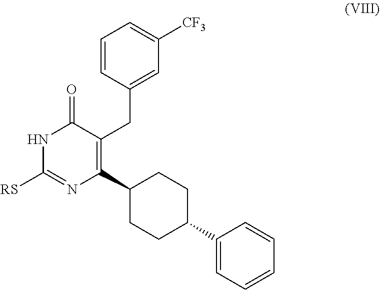

wherein R is C₁₋₁₂ alkyl.

In some embodiments, the compound of Formula VIII is the compound wherein R is C₆₋₁₂ alkyl. In some embodiments, the compound of Formula VIII is the compound wherein R is n-hexyl, n-heptyl, n-octyl, n-nonyl, or n-decyl. In some embodiments, the compound of Formula VIII is the compound wherein R is n-octyl.

In some embodiments, the compound of Formula VIII has the structure:

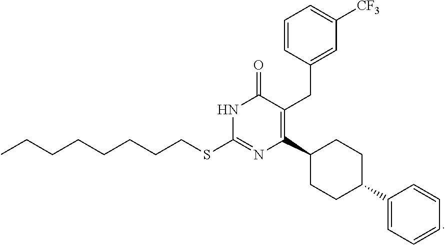 [SMILES: CCCCCCCCSc1nc([C@H]2CC[C@H](c3ccccc3)CC2)c(Cc2cccc(C(F)(F)F)c2)c(=O)[nH]1 ; "."]

The compound of Formula VIII is named 2-(octylthio)-6-((1r,4r)-4-phenylcyclohexyl)-5-(3-(trifluoromethyl)benzyl)pyrimidin-4(3H)-one using IUPAC nomenclature.

### IV. EXAMPLES

The following acronyms and abbreviations are used in the methods below:

| | |
|---|---|
| AcOH | Acetic acid |
| aq | Aqueous |
| atm | Atmospheric pressure |
| ° C. | Degree Celsius |
| DBU | 1,8-Diazabicyclo[5.4.0]undec-7-ene |
| DCM | Dichloromethane |
| DMF | N,N-Dimethylformamide |
| DMAP | N,N-Dimethylaminopyridine/4- |
| | (Dimethylamino)pyridine |
| DSC | Differential Scanning Calorimetry |
| EDC | 1-Ethyl-3-(3-dimethylaminopropyl)carbodiimide |
| eq | Equivalent |
| GF | Glass microfiber filter paper |
| g | Gram |
| HCl | Hydrochloric acid |
| hdpe | High density polyethylene |
| HPLC | High-performance Liquid Chromatography |
| KF | Karl Fischer titration |
| L | Liter |
| LCAP | HPLC Area Percent |
| LCWP | HPLC Weight Percent |
| M | Molar |
| MeCN | Acrylonitrile |
| MeOH | Methanol |
| mins | Minutes |
| mL | Milliliter |
| nBuOH | butan-1-ol |
| NMP | N-Methyl-2-pyrrolidone |
| Pd—C | Palladium on carbon |
| ¹H-NMR | Proton Nuclear Magnetic Resonance |
| ref std | Reference Standard |
| RT | Room Temperature |
| SM | Starting Material |
| T | Temperature |
| THF | Tetrahydrofuran |
| TMS | Tetramethylsilane |
| TG-DTA | Thermogravimetric Differential Thermal Analysis |
| vol | Volume |
| wrt | With respect to |
| Wt | Weight |
| XRPD | X-ray Powder Diffraction |

X-ray Powder Diffraction (XRPD). XRPD analyses were performed using a Panalytical Xpert Pro diffractometer equipped with a Cu X-ray tube and a Pixcel detector system. The samples were analyzed at ambient temperature in transmission mode and held between PVC films. The default XRPD program was used (range 3-40° 20, step size 0.013°, counting time 99 sec, ˜22 min run time/counting time 49 sec for the Compound of Formula II and ˜11 min run time/counting time 22 sec for the both Forms of the Compound of Formula VII Samples were spun at 60 rpm during data collection. XRPD patterns were sorted, manipulated using HighScore Plus 2.2c software.

Differential Scanning calorimetry (DSC). DSC analyses were carried out on a Perkin Elmer Jade Differential Scanning calorimeter. Accurately weighed samples were placed in gold pans and lid secured. Each sample was heated under nitrogen at a rate of 5° C./minute to a maximum of 200 or 300° C.

Thermogravimetric Differential Thermal Analysis (TG-DTA). Thermogravimetric (TG) analyses were carried out on a Mettler Toledo TGA/DSC 1 STARe simultaneous thermal analysis instrument. Samples were placed in an aluminium sample pan, inserted into the TG furnace and accurately weighed. Under a stream of nitrogen at a rate of 10° C./minute, the heat flow signal was stabilized for one minute at 30° C., prior to heating to 300° C.

Proton Nuclear Magnetic Resonance Spectroscopy (1H-NMR). Proton NMR analyses were performed on a 500 MHz Bruker AVANCE NEO instrument fitted with a Prodigy BBO CryoProbe. Samples were dissolved in the appropriate deuterated NMR solvent containing tetramethylsilane (TMS) as an internal standard, possessing an isotopic purity of ≥99.5 atom % D, then the solution of the sample was analyzed using 5 mm Virgin NMR tubes.

“Conditions suitable” for conducting the methods of the present invention include the time and temperature for conducting the methods, as defined below.

The reaction steps of the present invention can be performed for any suitable reaction time. For example, the reaction time can be for minutes, hours, or days. In some embodiments, the reaction time can be for several hours, such as at least eight hours. In some embodiments, the reaction time can be for several hours, such as at least overnight. In some embodiments, the reaction time can be for several days. In some embodiments, the reaction time can be for at least two hours. In some embodiments, the reaction time can be for at least eight hours. In some embodiments, the reaction time can be for at least several days. In some embodiments, the reaction time can be for about two hours, or for about 4 hours, or for about 6 hours, or for about 8 hours, or for about 10 hours, or for about 12 hours, or for about 14 hours, or for about 16 hours, or for about 18 hours, or for about 20 hours, or for about 22 hours, or for about 24 hours. In some embodiments, the reaction time can be for about 1 day, or for about two days, or for about three days, or for about four days, or for about five days, or for about six days, or for about a week, or for about more than a week.

The reaction steps of the present invention can be performed at any suitable reaction temperature. Representative temperatures include, but are not limited to, below room temperature, at room temperature, or above room temperature. Other temperatures useful in the methods of the present invention include from about −40° C. to about 65° C., or from about room temperature to about 40° C., or from about 40° C. to about 65° C., or from about 40° C. to about 60° C. In some embodiments, the reaction mixture can be at a temperature of about room temperature, or at a temperature of about 15° C., or at about 20° C., or at about 25° C. or at about 30° C., or at about 35° C., or at about 40° C., or at about 45° C., or at about 50° C., or at about 55° C., or at about 60° C., or at about 65° C.

### Example 1. Preparation of 2-(octylthio)-6-((1r,4r)-4-phenylcyclohexyl)-5-(3-(trifluoromethyl)benzyl)pyrimidin-4(3H)-one (Formula VIII)

Preparation of 6-((1r,4r)-4-phenylcyclohexyl)-2-thioxo-5-(3-(trifluoromethyl)benzyl)-2,3-dihydropyrimidin-4 (1H)-one (Formula IX)

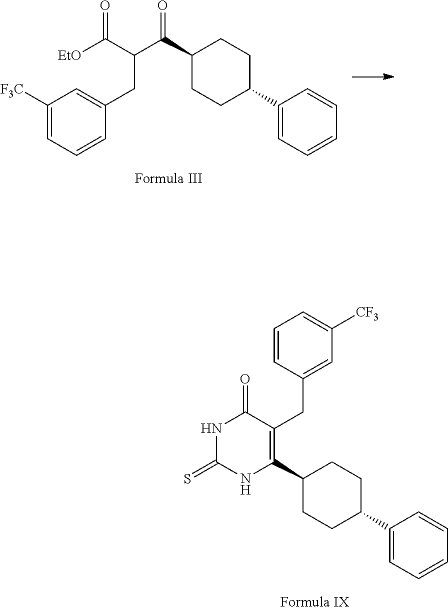 [SMILES: CCOC(=O)C(Cc1cccc(C(F)(F)F)c1)C(=O)[C@H]1CC[C@H](c2ccccc2)CC1 ; O=c1[nH]c(=S)[nH]c([C@H]2CC[C@H](c3ccccc3)CC2)c1Cc1cccc(C(F)(F)F)c1]

Formula III was prepared according to the methods described in WO2022/140293.

Formula III (58.0 g, 134.1 mmol, 1 eq.) and thiourea (61.2 g, 804.7 mmol, 6 eq.) were slurried in DMF (5 vol., 290 ml). The contents were stirred under N₂ until a solution was formed. DBU (51.0 g, 50.1 ml, 335.3 mmol, 2.5 eq.) was added and the contents warmed to 80° C. The stream was aged overnight at 80° C. then analysed via HPLC for consumption of Formula III (<2LCAP Formula III remaining). MeCN (3 vol., 174 ml) was added (to solubilize solids and avoid gumming after addition of acid), followed by 1M HCl (3.5 vol., 203 ml) added over ˜1 hour generating an initially fine slurry, that slowly transformed to a thicker mobile white slurry. Liquor loss of the slurry was determined by HPLC as 0.5 mg/ml and the product was isolated via filtration. The wet cake was washed sequentially with MeCN (3 vol., 174 ml), 1:1 MeCN:water (3 vol., 174 ml) and water (3 vol., 174 ml). The product was dried under vacuum at 50° C. overnight. Thiouracil Formula IX (42.5 g) was isolated as a white crystalline solid.

The characterization data of the title product matched that previously described.

Preparation of 2-(octylthio)-6-((1r,4r)-4-phenylcyclohexyl)-5-(3-(trifluoromethyl)benzyl)pyrimidin-4 (3H)-one (Formula VIII)

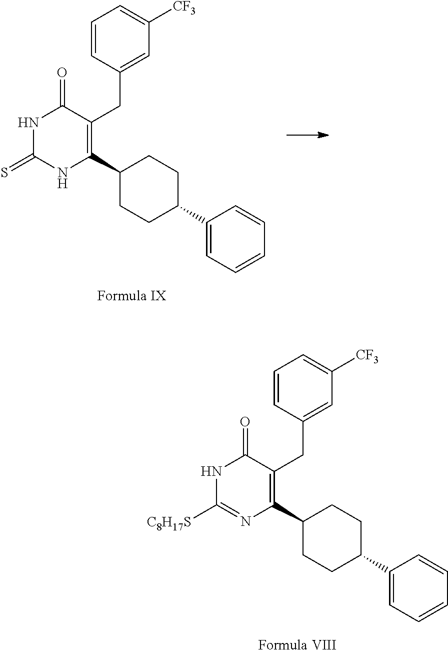

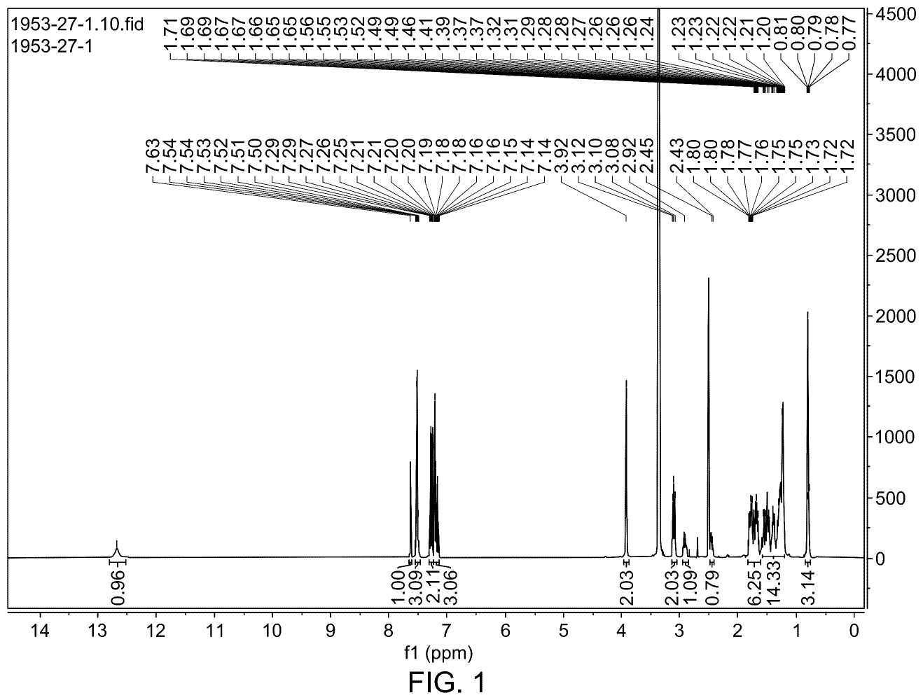

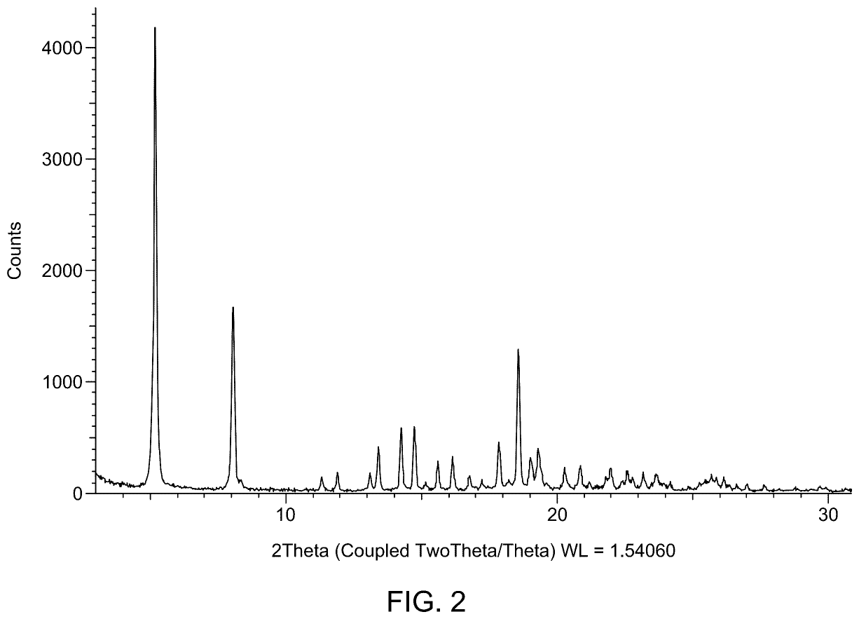

Thiouracil Formula IX (41 g, 92.2 mmol, 1 eq.) and K₂CO₃ (12.7 g, 92.2 mmol, 1 eq.) were slurried in NMP (410 ml, 10 vol.) under N₂. After ageing for 30 minutes, 1-iodooctane (22.1 g, 16.6 ml, 92.2 mmol, 1 eq.) was added dropwise over ˜15 minutes. The mixture was aged at 25° C. for 4 hours. HPLC indicated complete consumption of thiouracil Formula IX. The mixture was treated with MeCN (3 vol., 123 ml) and water (13 vol., 533 ml) added over ˜1 hour, generating a mobile white slurry. Liquor loss of the slurry was determined by HPLC as 0.98 mg/ml and the product was isolated via filtration. The product was washed sequentially with MeCN (3 vol., 123 ml), 1:1 MeCN:water (3 vol., 123 ml) and water (3 vol., 123 ml). After drying under suction for 1 hour, the wet cake was re-slurried in MeCN:water (4:1, 3 vol., 123 ml) and aged overnight. The following morning the slurry was filtered and washed with water (3 vol., 123 ml). The product was dried under vacuum at 50° C. overnight. Sulfide Formula VIII (47.3 g) was isolated as a crystalline white solid. m/z [M+H]: 557.4. 1H NMR (DMSO-d6) is provided in FIG. 1. The XRPD is provided in FIG. 2.

### Example 2. Oxone Preparation of 6-((1r,4r)-4-phenylcyclohexyl)-5-(3-(trifluoromethyl)benzyl)pyrimidine-2,4 (1H,3H)-dione (Formula I)

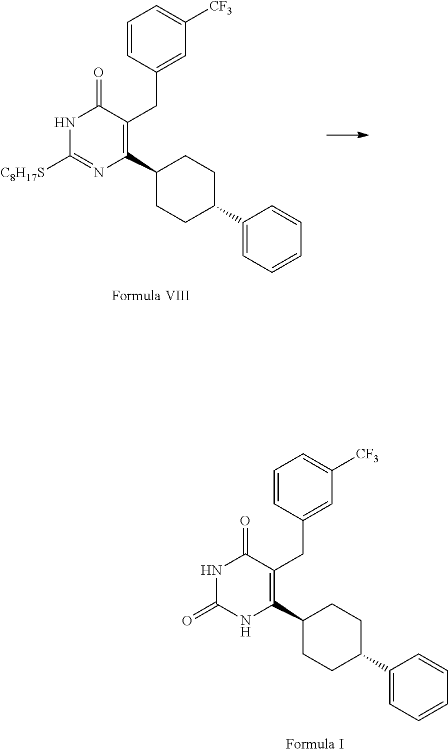

A 2 L vessel was rinsed with NMP prior to use. Formula VIII (39.0 g, 69.9 mmol, 1.0 equiv.) and OXONE® (107.4 g, 349.6 mmol, 5.0 equiv.) were charged to the vessel and the vessel purged with a positive pressure of nitrogen for 15 mins. NMP (390 mL, 10 vol.) was charged, and the vessel jacket was heated to 80° C. The reaction mixture was aged for 4 h.

H₂O (19.5 mL, 0.5 vol.) was charged, and the reaction mixture was aged for 17 h. MeCN (117 mL, 3 vol.) was charged. H₂O (273 mL, 7.5 vol.) was charged slowly over 0.5 h. The resulting slurry was aged for 0.5 h after which a sample was taken to measure liquor concentration by HPLC. The slurry was filtered, then the filter cake was washed with MeCN (117 mL, 3 vol.), 1:1 MeCN/H₂O (117 mL, 3 vol.) and H₂O (117 mL, 3 vol.) and dried under vacuum for 15 mins. The wet cake and H₂O (390 mL, 10 vol.) were charged to the vessel and the contents aged 80° C. for 1 h. The slurry was filtered, the cake was washed with H₂O (3×150 mL, 3×3 vol.) and dried under vacuum for 15 mins. The cake was dried under vacuum at 50° C. for 72 h and analysed for Wt %. 24.0 g of isolated Formula I obtained corresponding to an 80% yield.

Formula I (23.9 g, 55.8 mmol) was charged to a 500 mL 3-necked round-bottomed flask, fitted with overhead stirring. THF (155 mL, 6.5 vol.) was charged to the vessel and the vessel was heated to 60° C. at which point, Formula I was in solution. The vessel was cooled to RT. At≈40° C. seed (50 mg) was charged. Once the target temperature was reached (20° C.), 1-2 volumes of heptane was charged and the mixture was aged for 0.5 h, then the slurry was sampled and filtered. The resulting filtrate was analyzed by HPLC to determine the product content in the filtrate. This process was repeated until HPLC analysis of the resulting mother liquor was approximately 1.33 mg/mL. After a total of 8 volumes of heptane was added, the slurry was filtered, then washed with 3:4 THF/heptane (72 mL, 3 vol.), heptane (2×72 mL, 2×3 vol.). The slurry was filtered, then washed with 3:4 THF/heptane (72 mL, 3 vol.), heptane (2×72 mL, 2×3 vol.).

The cake was dried under vacuum at 50° C. for 72 h and analysed for Wt %. 22.9 g of Formula I was obtained as a white crystalline solid, corresponding to 96% yield for the recrystallisation (77% yield overall for the isolation of Formula I).

Karl-Fisher titration detected 3.21 ppm of H₂O was present in Formula I.

The characterization data of the title product matched those of Example 6 of U.S. Pat. No. 8,685,973, and Example 2 of U.S. Pat. No. 11,548,856.

### Example 3. Hydrogen Peroxide Preparation of 6-((1r,4r)-4-phenylcyclohexyl)-5-(3-(trifluoromethyl)benzyl)pyrimidine-2,4 (1H,3H)-dione (Formula I)

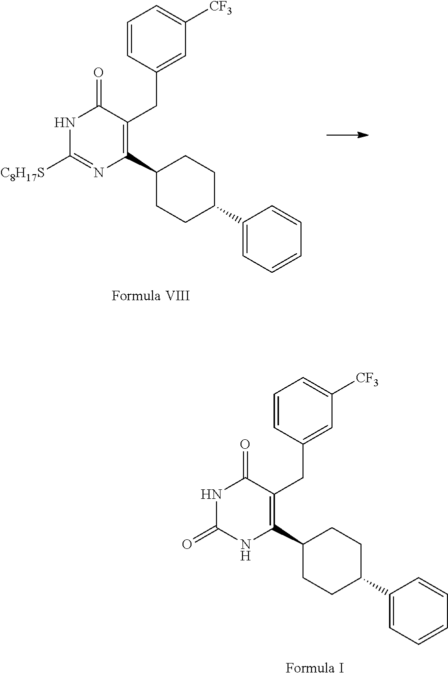

Octyl sulphide intermediate (Formula VIII, 8 g, 14.36 mmol, 1 eq) was dissolved in NMP (5 vol, 40 mL). The solution was treated with H₂SO₄ (3 eq, 2.3 ml, 4.2 g, 43.1 mmol). The contents were heated to 80° C. To the solution was added H₂O₂ (50% in water, 3 eq, 2.44 ml, 2.93 g, 43.1 mmol) over a period of 3 hours via syringe pump. The contents were aged overnight at 80° C. After aging, the contents were cooled to 20° C. and the stream analyzed for assay yield (89% product observed).

The solution was treated with MeCN (3 vols, 24 mls), then water (7.5 vols, 60 mls) added over 1 hour. The subsequent slurry was aged overnight. The product was isolated via filtration and washed sequentially with MeCN (3 vols, 24 mL), 1:1 MeCN:water (3 vols, 24 L) and water (3 vols, 24 L). The crude product was re-slurried in water (10 vol, 80 mL), and warmed to 80° C. for 1 hour. The slurry was cooled to 20° C. and aged for 1 hour. The product was isolated via filtration and washed with water (3×3 vols, 24 mL each). The product was transferred to a vacuum oven, and dried overnight at 50° C. Yield=4.85 g (79%); LCWP=98.2%.

The characterization data of the title product matched those of Example 6 of U.S. Pat. No. 8,685,973, and Example 2 of U.S. Pat. No. 11,548,856.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method of preparing a compound of Formula I: 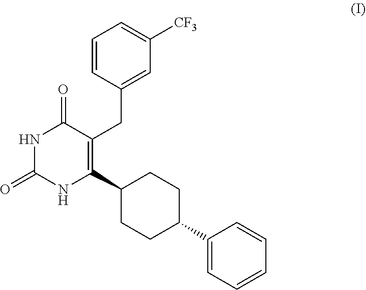 [SMILES: O=c1[nH]c(=O)c(Cc2cccc(C(F)(F)F)c2)c([C@H]2CC[C@H](c3ccccc3)CC2)[nH]1]
the method comprising:
(a) forming a first reaction mixture comprising a first solvent, a compound of Formula VIII: 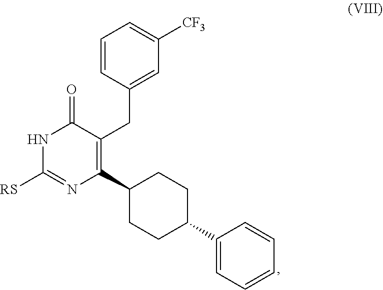
and an oxidizing agent, under conditions suitable to prepare the compound of Formula I,
wherein R is C₁₋₁₂ alkyl.

2. The method of claim 1, wherein the first solvent comprises acetone, methyl acetate, ethyl acetate, isopropyl acetate, N,N-dimethylformamide (DMF), N,N-dimethylacetamide (DMAc), dimethylsulfoxide (DMSO), 2-methyltetrahydrofuran (2-MeTHF), tetrahydrofuran (THF), N-methyl-2-pyrrolidone (NMP), or combinations thereof.

3. (canceled)

4. The method of claim 1, wherein R is C₆₋₁₂ alkyl.

5.-6. (canceled)

7. The method of to claim 1, wherein the compound of Formula VIII has the structure:

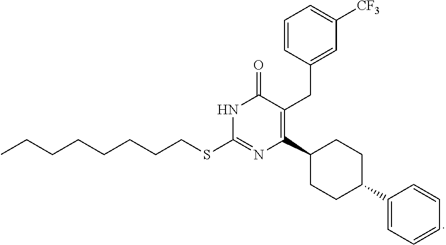 [SMILES: CCCCCCCCSc1nc([C@H]2CC[C@H](c3ccccc3)CC2)c(Cc2cccc(C(F)(F)F)c2)c(=O)[nH]1 ; "."]

8. The method of claim 1, wherein the oxidizing agent comprises potassium peroxymonosulfate (OXONE®), hydrogen peroxide, nitric acid, potassium chlorate, sulfuric acid, peroxydisulfuric acid, hypochlorite, potassium permanganate, or combinations thereof.

9-13. (canceled)

14. The method of claim 11, wherein the first reaction mixture further comprises a strong acid.

15. The method of claim 14, wherein the strong acid comprises trifluoroacetic acid, trichloroacetic acid, ethane-1,2-disulfonic acid, naphthalene-2-sulfonic acid, naphthalene-1,5-disulfonic acid, hydrofluoric acid, hydrochloric acid, hydrobromic acid, hypochlorous acid, chloric acid, perchloric acid, sulfuric acid, nitric acid, phosphoric acid, methanesulfonic acid, ethanesulfonic acid, benzenesulfonic acid, p-toluenesulfonic acid, trifluoromethanesulfonic acid, camphorsulfonic acid, or combinations thereof.

16.-17. (canceled)

18. A method of preparing a compound of Formula VIII: 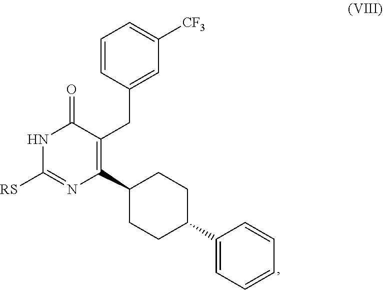
the method comprising:
(b) forming a second reaction mixture comprising an alkylating agent, a second non-nucleophilic base, a second solvent, and a compound of Formula IX: 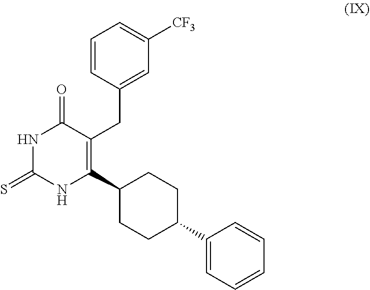 [SMILES: O=c1[nH]c(=S)[nH]c([C@H]2CC[C@H](c3ccccc3)CC2)c1Cc1cccc(C(F)(F)F)c1]
under conditions suitable to form the compound of Formula VIII,
wherein R is C₁₋₁₂ alkyl;
the alkylating agent is C₁₋₁₂ alkyl-halide or C₁₋₁₂ alkyl-OS(O)2R^{a};
R^{a} is C₁₋₆ alkyl, C₁₋₆ haloalkyl, and phenyl substituted with 0, 1, 2 or 3 R^{a1}; and
each R^{a1} is independently C₁₋₆ alkyl, halogen, or C₁₋₆ haloalkyl.

19. The method of claim 18, wherein the alkylating agent is C₁₋₁₂ alkyl-halide.

20. The method of claim 18, wherein the second reaction mixture comprises a C₆₋₁₂ alkyl-iodide.

21.-24. (canceled)

25. The method of claim 18, wherein the second non-nucleophilic base comprises sodium carbonate, potassium carbonate, cesium carbonate, triethylamine, N,N-diisopropyl ethylamine (DIPEA), N,N-dimethyl isopropylamine (DIMPA), piperidine, 1-ethylpiperidine, N-methylmorpholine, N-methylpyrrolidine, N,N-dimethylamine, piperazine, N-methylpiperazine, pyridine, N,N-dimethylaniline, N,N-diethylaniline, 2,6-lutidine, 2,4,6-collidine, 4-dimethyl aminopyridine (DMAP), quinuclidine, 4-pyrrolidinopyridine, 1,5-diazabicyclo(4.3.0) non-5-ene (DBN), 1,8-diazabicyclo[5.4.0]undec-7-ene (DBU), 1,4-diazabicyclo[2.2.2]octane (DABCO), or combinations thereof.

26-32. (canceled)

33. A method of preparing a compound of Formula IX: 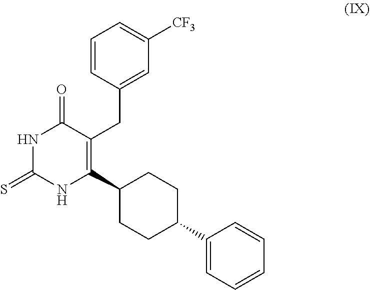 [SMILES: O=c1[nH]c(=S)[nH]c([C@H]2CC[C@H](c3ccccc3)CC2)c1Cc1cccc(C(F)(F)F)c1]
the method comprising:
(c) forming a third reaction mixture comprising a thiourea, a third non-nucleophilic base, a third solvent, and a compound of Formula III: 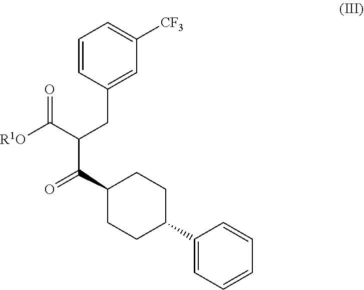
under conditions suitable to prepare the compound of Formula IX,
wherein R¹ is C₁₋₆ alkyl.

34. The method of claim 33, wherein R¹ is methyl, ethyl or n-propyl.

35. The method of claim 33, wherein the compound of Formula III has the structure:

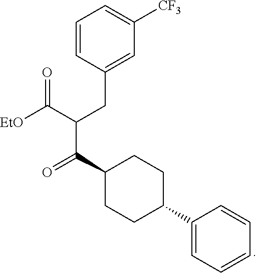 [SMILES: CCOC(=O)C(Cc1cccc(C(F)(F)F)c1)C(=O)[C@H]1CC[C@H](c2ccccc2)CC1 ; "."]

36. The method of any claim 3, wherein the third solvent comprises acetone, methyl acetate, ethyl acetate, isopropyl acetate, N,N-dimethylformamide (DMF), N,N-dimethylacetamide (DMAc), dimethylsulfoxide (DMSO), 2-methyltetrahydrofuran (2-MeTHF), tetrahydrofuran (THF), N-methyl-2-pyrrolidone (NMP), or combinations thereof.

37. (canceled)

38. The method of claim 33, wherein the third non-nucleophilic base comprises triethylamine, N,N-diisopropyl ethylamine (DIPEA), N,N-dimethyl isopropylamine (DIMPA), piperidine, 1-ethylpiperidine, N-methylmorpholine, N-methylpyrrolidine, N,N-dimethylamine, piperazine, N-methylpiperazine, pyridine, N,N-dimethylaniline, N,N-diethylaniline, 2,6-lutidine, 2,4,6-collidine, 4-dimethyl aminopyridine (DMAP), quinuclidine, 4-pyrrolidinopyridine, 1,5-diazabicyclo(4.3.0)non-5-ene (DBN), 1,8-diazabicyclo[5.4.0]undec-7-ene (DBU), 1,4-diazabicyclo[2.2.2]octane (DABCO), or combinations thereof.

39.-43. (canceled)

44. The method of claim 1, comprising
(c) forming the third reaction mixture comprising thiourea, DBU in an amount of about 2.5 molar equivalents to the compound of Formula III, N,N-dimethylformamide, and the compound of Formula III, under conditions suitable to prepare the compound of Formula IX;
(b) forming the second reaction mixture comprising n-octyl-iodide in an amount of about 1 molar equivalent to the compound of Formula IX, potassium carbonate in an amount of about 1 molar equivalent to the compound of Formula IX, N-methyl-2-pyrrolidone, and the compound of Formula IX, under conditions suitable to form the compound of Formula VIII; and
(a) forming the first reaction mixture comprising potassium peroxomonosulfate (oxone), N-methyl-2-pyrrolidone, and the compound of Formula VIII, under conditions suitable to prepare the compound of Formula I.

45. The method of claim 1, comprising
(c) forming the third reaction mixture comprising thiourea, DBU in an amount of about 2.5 molar equivalents to the compound of Formula III, N,N-dimethylformamide, and the compound of Formula III, under conditions suitable to prepare the compound of Formula IX;
(b) forming the second reaction mixture comprising n-octyl-iodide in an amount of about 1 molar equivalent to the compound of Formula IX, potassium carbonate in an amount of about 1 molar equivalent to the compound of Formula IX, N-methyl-2-pyrrolidone, and the compound of Formula IX, under conditions suitable to form the compound of Formula VIII; and
(a) forming the first reaction mixture comprising hydrogen peroxide in an amount of from 3 to 7 molar equivalents to the compound of Formula VIII, sulfuric acid, N-methyl-2-pyrrolidone, and the compound of Formula VIII, under conditions suitable to prepare the compound of Formula I.

46. A compound of Formula VIII: 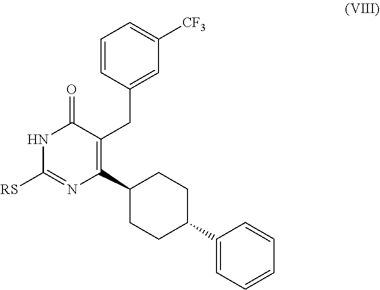
wherein R is C₁₋₁₂ alkyl.

47. The compound of claim 46, wherein R is C₆₋₁₂ alkyl.

48-49. (canceled)

50. The compound of claim 46, wherein the compound of Formula VIII has the structure: 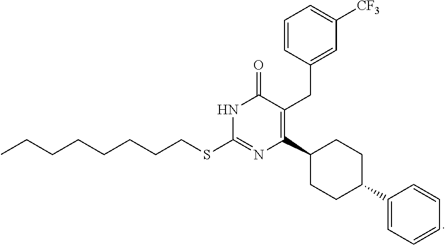 [SMILES: CCCCCCCCSc1nc([C@H]2CC[C@H](c3ccccc3)CC2)c(Cc2cccc(C(F)(F)F)c2)c(=O)[nH]1 ; "."]
